(19) United States
(12) Patent Application Publication
(10) Pub. No.: US 2021/0069223 A1
(43) Pub. Date: Mar. 11, 2021

(54) USE OF COMPOUND FOR PREVENTING AND/OR TREATING OBESITY

(71) Applicant: TCI CO., LTD., Taipei (TW)

(72) Inventor: YUNG-HSIANG LIN, Taipei (TW)

(21) Appl. No.: 17/016,384

(22) Filed: Sep. 10, 2020

Related U.S. Application Data
(60) Provisional application No. 62/898,018, filed on Sep. 10, 2019.

(30) Foreign Application Priority Data
Dec. 30, 2019 (TW) 108148432

(51) Int. Cl. A61K 31/7048; A61P 3/04

(52) U.S. Cl. CPC A61K 31/7048; A61P 3/04

(57) ABSTRACT

A method of preventing and/or treating obesity is provided. The method includes administering a composition to a subject, and the composition has at least one compound selected from the group consisting of 4-hydroxy-3-methoxycinnamic acid (also referred to as (E)-3-(4-hydroxy-3-methoxy-phenyl)prop-2-enoic acid), 7-[[2-O-(6-Deoxy-α-L-mannopyranosyl)-β-D-glucopyranosyl]]oxy]-2,3-dihydro-5-hydroxy-2-(4-hydroxyphenyl)-4H-1-benzopyran-4-one, and 5,7-Dihydroxy-2-(4-hydroxyphenyl)chroman-4-one. The compound can effectively promote lipolysis and/or reduce the cell fat content and can effectively reduce the fat content in cells to achieve the purpose of preventing and/or treating obesity.

### CROSS-REFERENCES TO RELATED APPLICATIONS

This application claims the benefit of U.S. provisional application Ser. No. 62/898,018, filed on Sep. 10, 2019 and claims the priority of Patent Application No. 108148432 filed in Taiwan, R.O.C. on Dec. 30, 2019. The entirety of the above-mentioned patent applications are hereby incorporated by references herein and made a part of the specification.

### BACKGROUND

### Technical Field

The present disclosure relates to a method of preventing and/or treating obesity in a subject which includes administering a composition to the subject. The composition has at least one compound selected from the group consisting of 4-hydroxy-3-methoxycinnamic acid (also referred to as (E)-3-(4-hydroxy-3-methoxy-phenyl)prop-2-enoic acid), 7-[[2-O-(6-Deoxy-α-L-mannopyranosyl)-β-D-glucopyranosyl]]oxy]-2,3-dihydro-5-hydroxy-2-(4-hydroxyphenyl)-4H-1-benzopyran-4-one, 5,7-Dihydroxy-2-(4-hydroxyphenyl)chroman-4-one, and any combination thereof.

### Related Art

In recent years, the incidence of global obesity has gradually increased, and the World Health Organization (WHO) described the situation of the rapidly rising obesity ratio as “infectious diseases”, and called it as “Globesity”. According to the estimation by the World Health Organization in 2014, about 39% of the global population were overweight (about 1.9 billion people), and 13% of the global population were obese (about 0.6 billion people). With the change of eating habits and the improvement of life quality, the obesity rate in Taiwan has increased year by year. According to the investigation on the change of national nutrition and health status published by the Health Promotion Administration of Ministry of Health and Welfare, the overweight or obesity rate in adults is as high as about forty-three percent, while the rates in males and in females are respectively forty-nine percent and forty percent. In other words, there is averagely one overweight or obese person in every two males in Taiwan, and averagely one overweight or obese person in every two to three females. In addition, more than 200,000 of them have reached the standard of morbid obesity, and have to be treated surgically.

Obese individuals may increase risks of various health problems, and cause various complications, including diseases such as hypertension, hyperlipidemia, cardiovascular diseases, sleep apnea, angina, degenerative arthritis, hyperuricemia osteoarthritis, type II diabetes and cancer. Due to excessive adipocytes in the body, harmful substances hindering the action of insulin can be secreted excessively, so that the function of the insulin is hindered. Thus, the rising blood glucose cannot be effectively controlled, thereby resulting in the symptom of hyperglycemia. If the body is in the condition of hyperglycemia over a long period of time, the pancreas will be tired of secreting more insulin to reduce the blood glucose. Once the pancreas is overloaded, the type II diabetes would be caused. In addition, the blood fat refers to fat in the blood, and mainly includes cholesterol and triglycerides. When the concentration of cholesterol or triglycerides circulating in the blood is higher than a normal value, the condition is referred to as hyperlipidemia. When the value of cholesterol or triglycerides is too high, or the concentration of high-density lipoprotein is too low, the condition is referred to as dyslipidemia. The hyperlipidemia may not only cause heart diseases, but also be closely related to chronic diseases such as stroke, hypertension, diabetes, nephropathy, etc.

Therefore, obesity may reduce the life quality and may lead to premature death, so the average life expectancy of a morbid obese patient is much shorter than that of a normal weight person. Although genes may be involved in the development of obesity, obesity epidemics are mainly caused by high-calorie diets and sedentary lifestyles. Thus, obesity and its complications can be prevented by changing the lifestyles.

Although the bodyweight reduction and obesity treatment can be realized by low calorie diets and regular exercises, these methods are difficult to implement and have limited efficacy mainly because of adaptive physiological mechanisms of maintaining energy storage in the body. In addition, some medicines (such as orlistat, phentermine, or topiramate) have been approved to be used in the treatment on long-term obesity. However, these medicines often cause serious side effects, thereby limiting the therapeutic efficacy and decreasing the patient compliance. Furthermore, it is indeed that the bodyweight can be greatly reduced by a bodyweight reduction operation, but this kind of intervention type operation is not suitable for all overweight people. On the basis of these reasons, there is indeed a need to study safe and effective ways to reduce the bodyweight and fat accumulation.

Based on the above, in response to the obesity resulting from life and diet habit changes of the modem people, the overall health problems caused by the obesity, and the improvement of the health care concept, it is necessary to develop a composition capable of effectively promoting lipolysis so as to reduce fat formation and reduce obesity chances, and a method of preventing or treating obesity in a subject.

### SUMMARY

Therefore, one objective of the present disclosure is to provide a method of preventing and/or treating obesity in a subject. The method includes administering a composition to the subject, and the composition includes at least one compound with therapeutically effective concentration selected from the group consisting of 4-hydroxy-3-methoxycinnamic acid (also referred to as (E)-3-(4-hydroxy-3-methoxy-phenyl)prop-2-enoic acid). 7-[[2-O-(6-Deoxy-α-L-mannopyranosyl)-β-D-gluco-pyranosyl]]oxy]-2,3-dihydro-5-hydroxy-2-(4-hydroxyphenyl)-4H-1-benzopyran-4-one, and 5,7-Dihydroxy-2-(4-hydroxyphenyl)chroman-4-one; and a pharmaceutically acceptable carrier.

Another objective of the present disclosure is to provide a method of promoting lipolysis. The method includes administering a composition to the subject, and the composition includes at least one compound with therapeutically effective concentration selected from the group consisting of 4-hydroxy-3-methoxycinnamic acid (also referred to as (E)-3-(4-hydroxy-3-methoxy-phenyl)prop-2-enoic acid), 7-[[2-O-(6-Deoxy-α-L-mannopyranosyl)-β-D-gluco-pyranosyl]]oxy]-2,3-dihydro-5-hydroxy-2-(4-hydroxyphenyl)-4H-1-benzopyran-4-one, and 5,7-Dihydroxy-2-(4-hydroxyphenyl)chroman-4-one; and a pharmaceutically acceptable carrier.

Another objective of the present disclosure is to provide a method of preventing or treating obesity in a subject. The method includes administering a therapeutically effective amount of 7-[[2-O-(6-Deoxy-α-L-mannopyranosyl)-β-D-gluco-pyranosyl]]oxy-2,3-dihydro-5-hydroxy-2-(4-hydroxyphenyl)-4H-1-benzopyran-4-one to the subject.

In an embodiment of the present disclosure, the therapeutically effective concentration of the compound is at least 10 μg/mL.

In another embodiment of the present disclosure, the pharmaceutical composition promotes lipolysis. In addition, the pharmaceutical composition promotes lipolysis in adipocytes.

In still another embodiment of the present disclosure, the therapeutically effective amount of the 7-[[2-O-(6-Deoxy-α-L-mannopyranosyl)-β-D-glucopyranosyl]]oxy]-2,3-dihydro-5-hydroxy-2-(4-hydroxyphenyl)-4H-1-benzopyran-4-one is at least 10 μg/mL.

In yet another embodiment of the present disclosure, the 7-[[2-O-(6-Deoxy-α-L-mannopyranosyl)-β-D-glucopyranosyl]]oxy]-2,3-dihydro-5-hydroxy-2-(4-hydroxyphenyl)-4H-1-benzopyran-4-one promotes lipolysis.

In the compounds of 4-hydroxy-3-methoxycinnamic acid (also referred to as (E)-3-(4-hydroxy-3-methoxy-phenyl)prop-2-enoic acid), 7-[[2-O-(6-Deoxy-α-L-mannopyranosyl)-β-D-glucopyranosyl]]oxy]-2,3-dihydro-5-hydroxy-2-(4-hydroxyphenyl)-4H-1-benzopyran-4-one, 5,7-Dihydroxy-2-(4-hydroxyphenyl)chroman-4-one, 5-hydroxy-2-(3-hydroxy-4-methoxy-phenyl)-4-oxo-3,4-dihydro-2h-chromen-7-yl 2-o-(6-deoxyhexopy-ranosyl) hexopyranoside, and (S)-2,3-Dihydro-5,7-dihydroxy-2-(3-hydroxy-4-methoxyphenyl)-4H-1-benzopyran-4-one of the present disclosure, the 4-hydroxy-3-methoxycinnamic acid (also referred to as (E)-3-(4-hydroxy-3-methoxy-phenyl)prop-2-enoic acid), the 7-[[2-O-(6-Deoxy-α-L-mannopyranosyl)-β-D-glucopyranosyl]]oxy]-2,3-dihydro-5-hydroxy-2-(4-hydroxyphenyl)-4H-1-benzopyran-4-one, and the 5,7-Dihydroxy-2-(4-hydroxyphenyl) chroman-4-one can effectively promote lipolysis to reduce the fat content in the cells (e.g. adipocytes). Therefore, the 4-hydroxy-3-methoxycinnamic acid (also referred to as (E)-3-(4-hydroxy-3-methoxy-phenyl)prop-2-enoic acid), the 7-[[2-O-(6-Deoxy-α-L-mannopyranosyl)-β-D-glucopyranosyl]]oxy]-2,3-dihydro-5-hydroxy-2-(4-hydroxyphenyl)-4H-1-benzopyran-4-one, and the 5,7-Dihydroxy-2-(4-hydroxyphenyl) chroman-4-one of the present disclosure can be used for promoting lipolysis and/or reducing the cell fat content. In addition, the composition may be a medicine, a health care product or a food capable of being applied to a subject by various ways, such as through oral administration, smearing, etc.

The following further describes the embodiments of the present disclosure with reference to accompanying drawings. The embodiments listed below are intended to clarify the present disclosure and are not intended to limit the scope of the present disclosure. A person skilled in the art may make some variations and modifications without departing from the spirit and scope of the present disclosure. Therefore, the protection scope of the present disclosure should be subject to the appended claims.

### BRIEF DESCRIPTION OF THE DRAWINGS

FIG. 1 is an HPLC fingerprint analysis chromatogram of a (Citrus aurantium extract according to an embodiment of the present disclosure.

FIG. 2 is a hydrogen spectrogram of a compound TCI-CA-0l purified from the Cirrus aurantium extract according to an embodiment of the present disclosure.

FIG. 3 is a mass spectrogram of the compound TCI-CA-01 purified from the Citrus aurantium extract according to an embodiment of the present disclosure.

FIG. 4 is a hydrogen spectrogram of a compound TCI-CA-02 purified from the Citrus aurantium extract according to an embodiment of the present disclosure.

FIG. 5 is a mass spectrogram of the compound TCI-CA-02 purified from the Citrus aurantium extract according to an embodiment of the present disclosure.

FIG. 6 is a hydrogen spectrogram of a compound TCI-CA-03 purified from the Citrus aurantium extract according to an embodiment of the present disclosure.

FIG. 7 is a mass spectrogram of the compound TCI-CA-03 purified from the Citrus aurantium extract according to an embodiment of the present disclosure.

FIG. 8 is a hydrogen spectrogram of a compound TC-CA-04 purified from the Citrus aurantium extract according to an embodiment of the present disclosure.

FIG. 9 is a mass spectrogram of the compound TCI-CA-04 purified from the Citrus aurantium extract according to an embodiment of the present disclosure.

FIG. 10 is a hydrogen spectrogram of a compound TCI-CA-05 purified from the Citrus aurantium extract according to an embodiment of the present disclosure.

FIG. 11 is a mass spectrogram of the compound TCI-CA-05 purified from the Citrus aurantium extract according to an embodiment of the present disclosure.

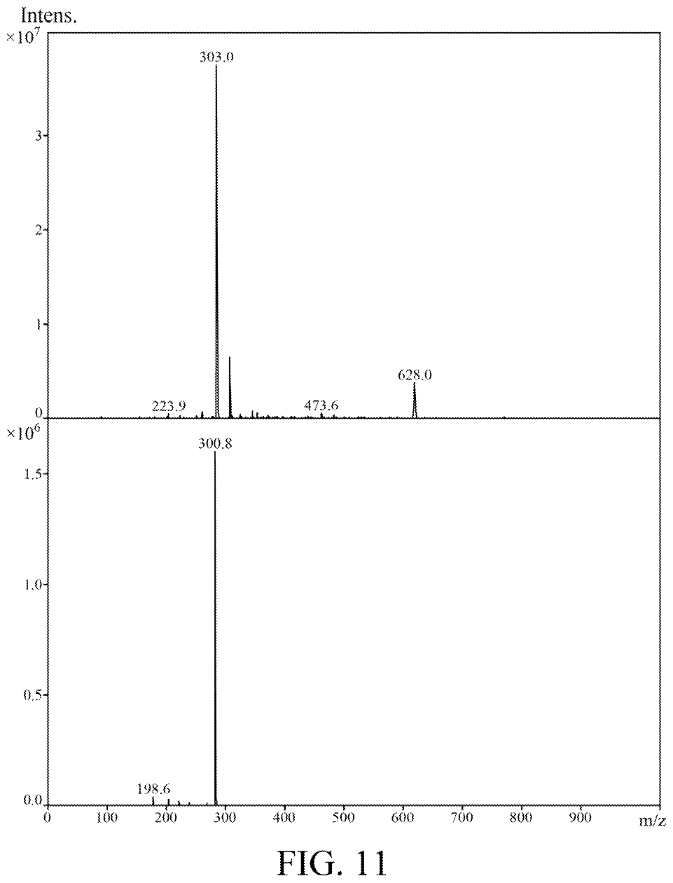

FIG. 12 is a histogram of efficacy of the compound TCI-CA-01, the compound TCI-CA-02, the compound TCI-CA-03, the compound TCI-CA-04 and the compound TCI-CA-05 purified from Citrus aurantium extract for promoting lipolysis according to an embodiment of the present disclosure. *** represents p<0.001.

### DETAILED DESCRIPTION

Values used herein are approximate values. All experiment data represents to be in a range of 20%, preferably in a range of 10%, and most preferably in a range of 5%.

Statistical analysis is performed by Excel software. The data is represented by mean value±standard deviation (SD). Differences between each group are analyzed by student's t-test.

In some embodiments of the present disclosure, the subject is in need of the prevention or treatment of obesity. The subject may be at risk due to genetic predisposition, diet, lifestyle, diseases, disorders, and the like. “Obesity” is a condition in which there is an excess of body fat. The operational definition of obesity is based on the Body Mass Index (BMI), which is calculated as body weight per height in meters squared (kg/m²). “Obesity” refers to a condition whereby a subject has a Body Mass Index (BMI) greater than or equal to 30 kg/m², or a condition whereby a subject has a BMI greater than or equal to 27 kg/m².

According to the present disclosure, a composition can be made into a dosage form suitable for being parenterally or orally administrated by a technology well known by those familiar to the skill. This includes, but is not limited to, an injection (such as a sterile aqueous solution or a dispersion), sterile powder, a tablet, a troche, a lozenge, a pill, a capsule, dispersible powder or granules, a solution, a suspension, an emulsion, syrup, elixir, slurry and an analogue thereof.

The composition according to the present disclosure can be administrated by a parenteral route selected from the group consisting of intraperitoneal injection, subcutaneous injection, intramuscular injection and intravenous injection.

The composition according to the present disclosure can include a pharmaceutically acceptable carrier widely used on a medicine manufacturing technology. For example, the pharmaceutically acceptable carrier can include one or more reagents selected from the group consisting of a solvent, an emulsifier, a suspending agent, a decomposer, a binding agent, an excipient, a stabilizing agent, a chelating agent, a diluent, a gelling agent, a preservative, a lubricant, an absorption delaying agent, a liposome and an analogue thereof. The selection and use and the quantity of the reagents fall within the professional skill and routine technical scope of those familiar to this technology.

According to the present disclosure, the pharmaceutically acceptable carrier may include at least one solvent selected from the group consisting of water, normal saline, phosphate buffered saline (PBS), and an alcohol containing aqueous solution.

According to the present disclosure, the composition can be administrated through a parenteral route selected from the group consisting of subcutaneous injection, intraepidermal injection, intradermal injection and intralesional injection.

According to the present disclosure, the composition can be used as a food additive to be added during raw material preparation by an existing method or added in a food manufacturing process, so as to be prepared with any kind of edible material into a food product for human beings and non-human animals to eat.

According to the present disclosure, types of the food product include but are not limited to beverages, fermented foods, bakery products, health foods and dietary supplements.

Bioactive ingredients (also can be referred to simply as “compounds”) in the present embodiment were separated and purified from Citrus aurantium, which is a Citrus evergreen shrub plant in Rutaceae, is also referred to as Suanchenghua, Qingcheng or Kucheng in Chinese. Citrus aurantium originates from Mediterranean, and had been used as a sterilizing agent of an aromatherapy method and a sedative of a phytotherapy method by the ancient Greece people during the ancient Greece times. The Cirrus aurantium prefers the warm and humid climate, and is not hardy. The flowers of the Citrus aurantium are white or have a burgundy color. It is known that the Cirrus aurantium has the efficacy of regulating Qi (kind of vital energy in Chinese culture) to resolve depression, relieving abdominal distension pain and vomiting, promoting blood circulation, relieving stagnant Qi to eliminate phlegm, etc. The Citrus aurantium has intense fragrance, which enables a smeller to forget fatigue, and thus the Citrus aurantium can be used for calming the mood and relieving nervousness.

In some embodiments, the Citrus aurantium refers to the flower of the Citrus aurantium. In some embodiments, the Citrus aurantium refers to a plant which not only includes the flower of the Citrus aurantium, but may also include other parts of the Citrus aurantium, such as its roots, stems, leaves, or fruits. In some embodiments, the Citrus aurantium may be fresh or dried.

The Citrus aurantium extract may be obtained by different ways. In some embodiments, whole plant of Citrus aurantium dry materials and extraction solvent (such as water, alcohol, or an alcohol and water mixed solution) are mixed according to a liquid-solid weight ratio of (5-20):(1-5). After homogenizing the solvent and the solid, the temperature of the solvent was raised to 50-100° C. and the Citrus aurantium was soaked in the solvent for 0.5-3 hours for conducting extraction. After the extraction, a raw extract solution (i.e. the solvent) was cooled to a room temperature. Then, the raw extract solution was filtered to obtain a filtrate. The filtrate was concentrated under reduced pressure at 45-70° C. to a condition that the Degrees Brix (° Bx) of the filtrate reached 11.5-12.5. Then, the Citrus aurantium extract of the present disclosure can be obtained.

According to the present disclosure, five kinds of compounds: [1]4-hydroxy-3-methoxycinnamic acid (also referred to as (E)-3-(4-hydroxy-3-methoxy-phenyl)prop-2-enoic acid) named as TCI-CA-01 herein, [2]7-[[2-O-(6-Deoxy-α-L-mannopyranosyl)-β-D-glucopyranosyl]]oxy]-2,3-dihydro-5-hydroxy-2-(4-hydroxyphenyl)-4H-1-benzopyran-4-one named as TCI-CA-02 herein, [3]5,7-Dihydroxy-2-(4-hydroxyphenyl) chroman-4-one named as TCI-CA-03 herein, [4]5-hydroxy-2-(3-hydroxy-4-methoxy-phenyl)-4-oxo-3,4-dihydro-2h-chromen-7-yl 2-o-(6-deoxyhexopy-ranosyl) hexopyranoside named as TCI-CA-04 herein, and [5](S)-2,3-Dihydro-5,7-dihydroxy-2-(3-hydroxy-4-methoxyphenyl)-4H-1-benzopyran-4-one named as TCI-CA-05 herein are purified from an ethyl acetate layer extract of Citrus aurantium by a column chromatography method and a thin layer chromatography (TLC) method.

According to the present disclosure, operation procedures, parameter conditions and the like about chemical separation and chemical structure analysis of the mixture fall within the professional skill and routine technical scope of those familiar to this technology.

The present disclosure provides a use of [1] 4-hydroxy-3-methoxycinnamic acid (also referred to as (E)-3-(4-hydroxy-3-methoxy-phenyl)prop-2-enoic acid), [2]7-[[2-O-(6-Deoxy-α-L-mannopyranosyl)-β-D-glucopyranosyl]]oxy]-2,3-dihydro-5-hydroxy-2-(4-hydroxyphenyl)-4H-1-benzopyran-4-one, and/or [3]5,7-Dihydroxy-2-(4-hydroxyphenyl)chroman-4-one for preparing a composition for promoting lipolysis and/or reducing the cell fat content. These compounds can effectively promote lipolysis, so as to reduce the fat content in the cells. Accordingly, the present disclosure provides a method of promoting lipolysis in a subject by administering the composition to the subject, and the composition includes at least one compound with therapeutically effective concentration selected from TCI-CA-01, TCI-CA-02 and TCI-CA-03. Since the method may promote lipolysis in a subject, the method can prevent and/or treat obesity in a subject as well.

The terms “therapeutically effective concentration” or “therapeutically effective amount” refer to a concentration or an amount that results in an improvement or remediation of the target disease, disorder, or symptoms of the disease or condition.

According to some embodiments of the present disclosure, the composition for promoting lipolysis and/or reducing the cell fat content can also include at least one compound having an effective concentration and selected from the group consisting of 4-hydroxy-3-methoxycinnamic acid (also referred to as (E)-3-(4-hydroxy-3-methoxy-phenyl)prop-2-enoic acid), 7-[[2-O-(6-Deoxy-α-L-mannopyranosyl)-β-D-glucopyranosyl]]oxy]-2,3-dihydro-5-hydroxy-2-(4-hydroxyphenyl)-4H-1-benzopyran-4-one, and 5,7-Dihydroxy-2-(4-hydroxyphenyl)chroman-4-one; and a pharmaceutically acceptable carrier. In some embodiments, the composition may be a medicine, a health care product or a food product.

Hereafter, detail extraction and purification methods of 4-hydroxy-3-methoxycinnamic acid (also referred to as (E)-3-(4-hydroxy-3-methoxy-phenyl)prop-2-enoic acid), 7-[[2-O-(6-Deoxy-α-L-mannopyranosyl)-β-D-glucopyranosyl]]oxy]-2,3-dihydro-5-hydroxy-2-(4-hydroxyphenyl)-4H-1-benzopyran-4-one, 5,7-Dihydroxy-2-(4-hydroxyphenyl) chroman-4-one, 5-hydroxy-2-(3-hydroxy-4-methoxy-phenyl)-4-oxo-3,4-dihydro-2h-chromen-7-yl 2-o-(6-deoxyhexopy-ranosyl) hexopyranoside, and (S)-2,3-Dihydro-5,7-dihydroxy-2-(3-hydroxy-4-methoxyphenyl)-4H-1-benzopyran-4-one of the present disclosure, and the test experiments of these five kinds of compounds for respectively promoting lipolysis in adipocytes will be illustrated in detail.

Chemical Analysis Material

n-hexane, ethyl acetate, acetone, methanol, ethanol, n-butanol, acetonitrile, Chloroform-dl (deuteration degree: 99.5%), methanol-d6 (deuteration degree: 99.5%), deuterium oxide (deuteration degree>99.8%), and dimethyl sulfoxide-d (deuteration degree>99.9%) are purchased from Merck company in Taiwan.

Chemical Analysis Instrument

A column chromatography method, a high performance liquid chromatography method (HPLC), and a thin layer chromatography (TLC) method were utilized for compounds separation. Specifically, column packing material used in the column chromatography were selected from, depending on the actual needs, Sephadex LH-20 (purchased from Amersham BioSciences, Britain), Diaion HP-20 (purchased from Mitsubishi Chemical Co., Japan), Merck Kieselgel 60 (40-63 μm, purchased from Merck, Germany. No.: Art. 9385), and Merck LiChroprep® RP-18 (40-63 μm, purchased from Merck, Germany, No.: Art. 0250). The high performance liquid chromatography system was provided with a Hitachi L-2310 series pump, a Hitachi L-2420 UV-VIS detector (detection wavelength: 200 nm to 380 nm), and D-2000 Elite data processing software. Column used in HPLC were selected from, depending on the actual needs, an analytical grade Discovery® HS C18 (250×4.6 mm, 5 μm; SUPELCO) column, a Mightysil RP-18 GP 250 (250×4.6 mm, 5 μm, purchased from Kanto Chemical) column, a semi-preparation grade Discovery® HS C18 (250×10.0 mm, 5 μm, purchased from SUPELCO) column and a preparation grade Discovery® HS C18 (250×21.0 mm, 5 μm, purchased from SUPELCO) column. The HPLC system was provided with ultraviolet lamps UVP UVGL-25 (wavelengths: 254 nm and 365 nm, which were the detection wavelength used in embodiments as well). A thin layer chromatography sheet was an aluminum sheet coated with silica gel 60 F₂₅₄ (0.25 mm, purchased from Merck, Germany) or RP-18 F₂₅₄₈ (0.25 mm, purchased from Merck, Germany).

Chemical structures of the compounds were analyzed by a mass spectrometry (MS) method and a nuclear magnetic resonance spectrometry (NMR) method. Specifically, a two-dimensional ion trap tandem Fourier transform mass spectrum (amaZon SL system, purchased from Bruker) was used for determination, and the unit was m/z. A NMR spectrometer (400 MHz Varian 400 FT-NMR) was used to obtain one-dimensional NMR spectrums. Chemical shift was represented by 6, and the unit is ppm. Tetramethysilane (TMS) was used as an internal standard product. A coupling constant (J) was measured in Hz. Symbol s represented singlet peak; symbol d represented doublet peak; symbol t represented triplet peak; symbol q represented quartet peak; symbol p represented quintet peak; symbol M represented multiplet peak; and symbol bras represented broad peak.

Unless specifically mentioned, the steps in the embodiments below are conducted under 1 atm and room temperature (25±5° C.).

### Embodiment 1 Separation and Purification of Compounds TCI-CA-01, TCI-CA-02, TCI-CA-03, TCI-CA-04 and TCI-CA-05 of the Present Disclosure

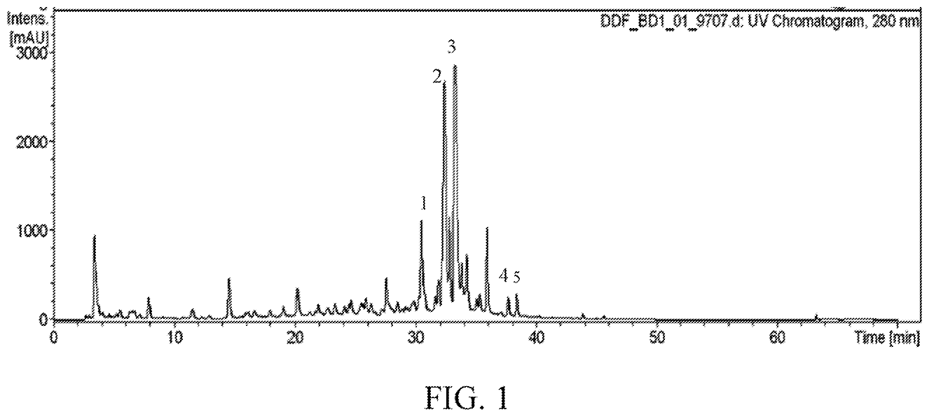

Firstly, a Citrus aurantium extract was prepared. The dried flowers of Citrus aurantium (purchased from Hangzhou botanical technology co., ltd) and water were mixed according to a liquid-solid weight ratio of 15:1. After homogenization, the temperature of the solvent was raised to and kept at 85° C., and the flowers of the Citrus aurantium was soaked in the solvent for 60 minutes for conducting extraction. After the extraction, the solvent was cooled to a room temperature. A raw extract (the solvent) was filtered by a 400-mesh filter to obtain a filtrate. Finally, the filtrate was concentrated under reduced pressure at 60° C. to a condition that the Degrees Brix (° Bx) of the filtrate reached 12.0, and then the Citrus aurantium extract of the present disclosure was obtained. The high performance liquid chromatography fingerprint chromatogram of the Citrus aurantium extract was as shown in FIG. 1 (column: Mightysil RP-18 GP 250 column; eluent: methanol; flow rate: 10 ml/minute).

Then, 10 L of the Citrus aurantium extract was taken for separation and purification. Ethyl acetate and the Citrus aurantium extract were mixed according to a volume ratio of 1:1 for extraction. After the Citrus aurantium extract was extracted for three times through this liquid-liquid distributive extraction method, the extraction liquid of the ethyl acetate layer was collected and merged, and was concentrated under reduced pressure to remove the solvent. Then, 4.4 g of the ethyl acetate layer extract of the Citrus aurantium extract was obtained.

Then, active ingredients in the ethyl acetate layer extract of the Citrus aurantium extract were traced by a bioassay guided fractionation method. Firstly, 4.4 g of the ethyl acetate layer extract was separated and purified by a column chromatography method. The column packing material was Sephadex LH-20, and methanol was used as an eluent. After the Citrus aurantium extract were separated into different fractions by the column, the different fractions were confirmed by their TLC pattern (developing solvent: the volume ratio of ethyl acetate and methanol is 9:1) and the fractions with similar pattern (or having the same target product) were combined with each other. Thus, 7 division layers (F1-F7) were obtained through the separation. Then, the division layer F1 (which is a layer including the fractions eluted out first) was further separated and purified by a medium pressure liquid chromatography (MPLC, CombiFlash® Rf™, purchased from Teledyne ISCO). Merck LiChroprep® RP-18 was filled in the column as a packing material. A mixed solution of water and methanol was used as an eluent, and the polarity was gradually reduced (in 60 minutes, linearly changed from 100% water to 100% methanol). The flow rate is 10 m/minute. After being separated into different fractions by the column, the different fractions were confirmed by their TLC pattern (developing solvent: the volume ratio of ethyl acetate and methanol is 9:1) and the fractions with similar pattern (or having the same target product) were combined with each other. Consequently, 5 sub-division layers (F1-1-F1-5) were obtained. The sub-division layers are labeled according to the order of being eluted out. That is, the sub-division layer F1-1 included the fraction eluted out first.

Then, the sub-division layer F1-3 was further separated and purified by the HPLC. A mixed solvent system of water and acetonitrile mixed according to a volume ratio of 4:1 was used as a mobile phase. The column was Discovery® HS C18 (250×4.6 mm, 5 μm; SUPELCO), and the flow rate was 2 ml/min. After separating the contents in different fractions and removing the mobile phase of the fractions, 6.9 mg of a compound TCI-CA-01, 8.0 mg of a compound TCI-CA-02, and 12.7 mg of a compound TCI-CA-03 were obtained.

Then, the sub-division layer F1-5 was further separated and purified by the HPLC. A mixed solvent system of water and acetonitrile mixed according to a volume ratio of 3:1 was used as a mobile phase. The column was Discovery® HS C18 (250×4.6 mm, 5 μm; SUPELCO), and the flow rate was 2 ml/min. After separating the contents in different fractions and removing the mobile phase of the fractions, 5.6 mg of a compound TCI-CA-04, and 9.2 mg of a compound TCI-CA-05 were obtained.

The compounds TCI-CA-01, TCI-CA-02, TCI-CA-03, TCI-CA-04, and TC-CA-05 were analyzed by a nuclear magnetic resonance spectrometer and a mass spectrometer and were then subjected to document and/or literature comparison to determine names and chemical structural formulas of the five compounds as shown in Table I below.

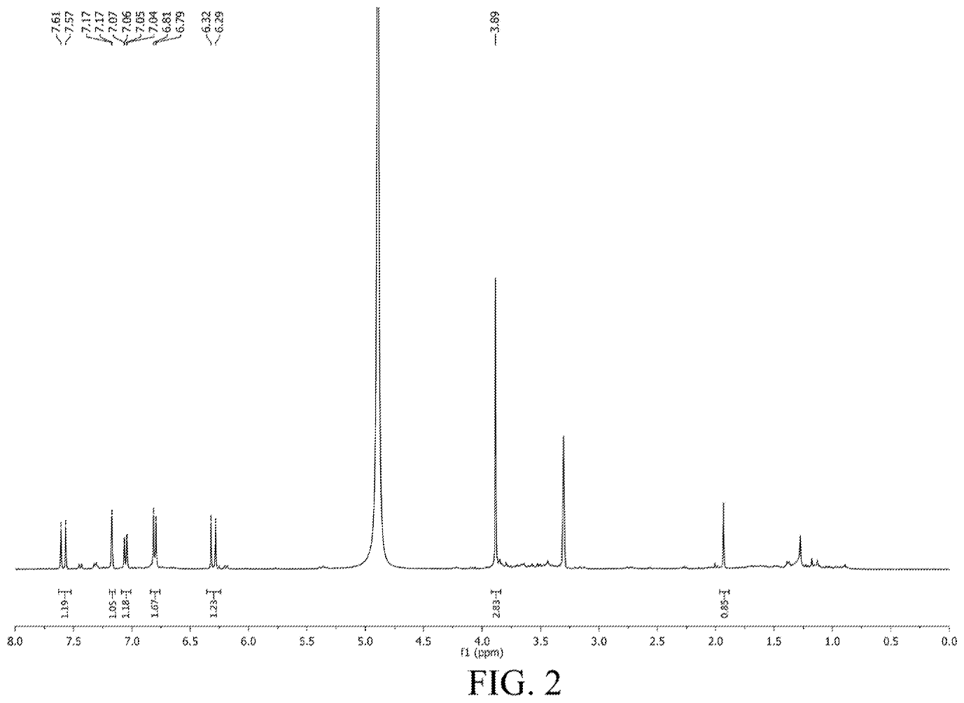

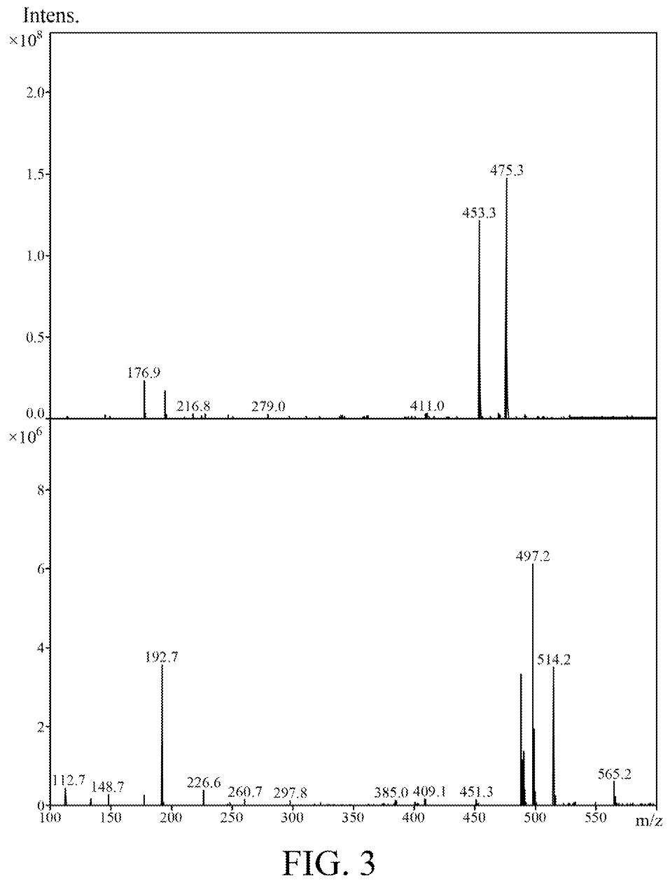

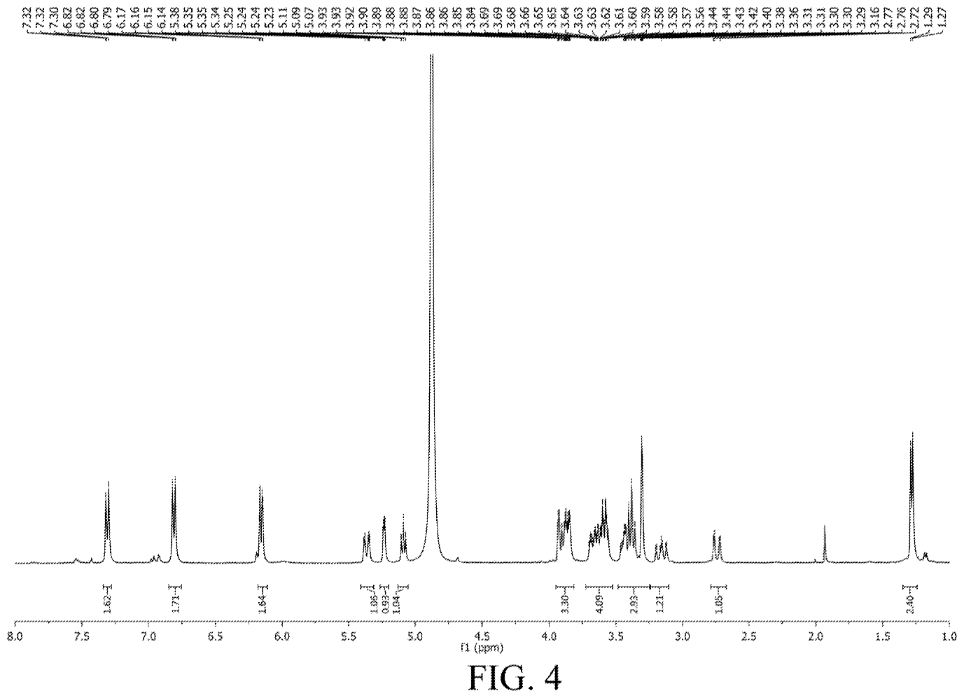

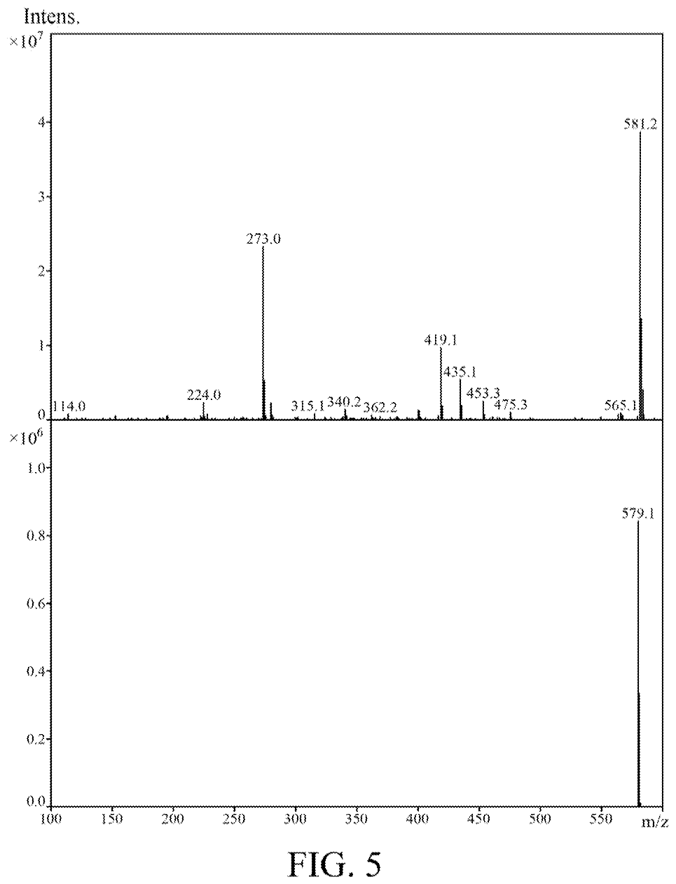

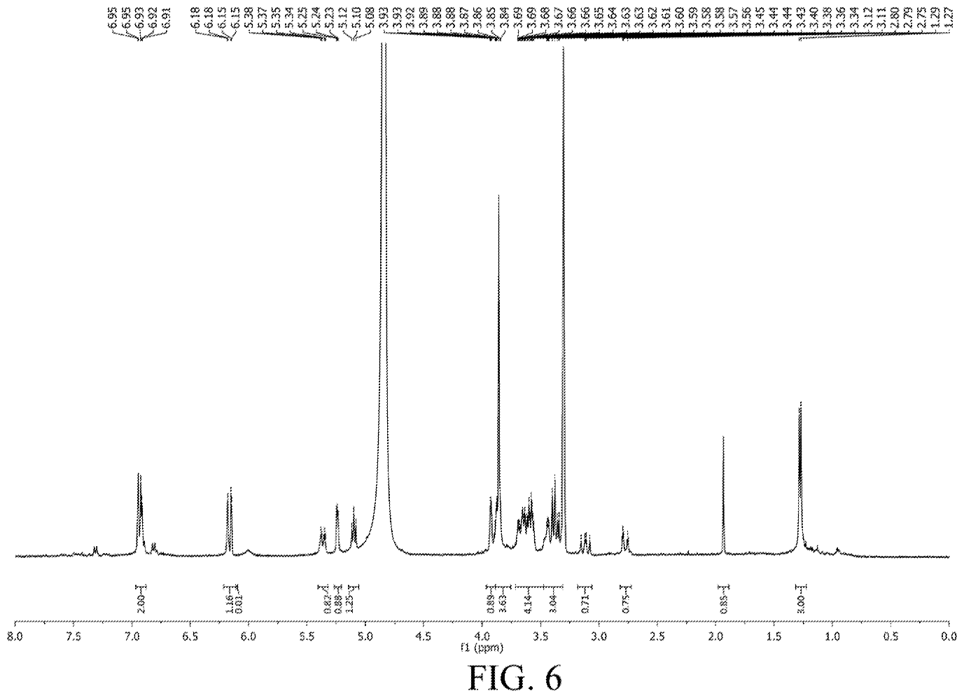

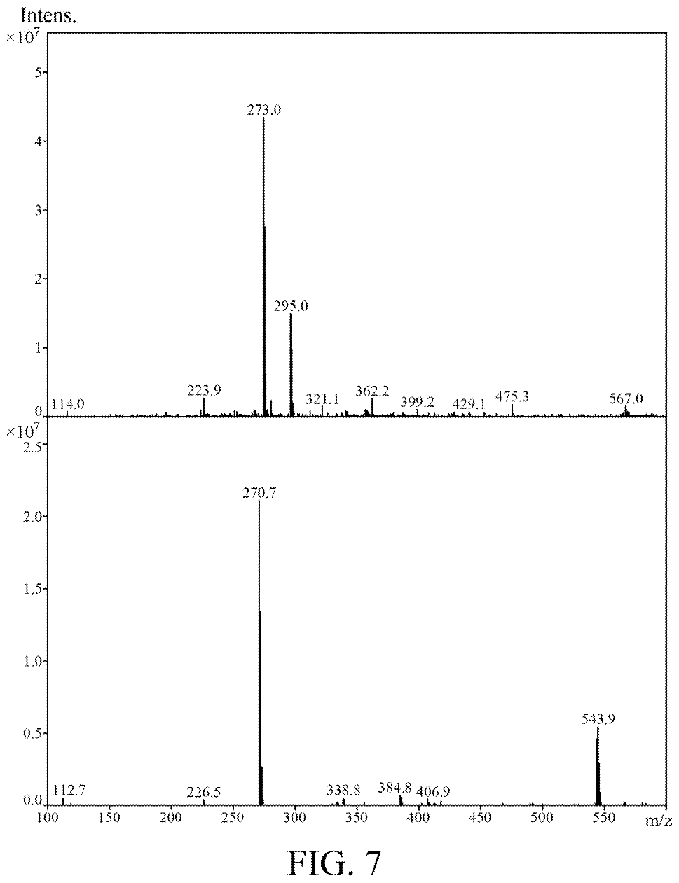

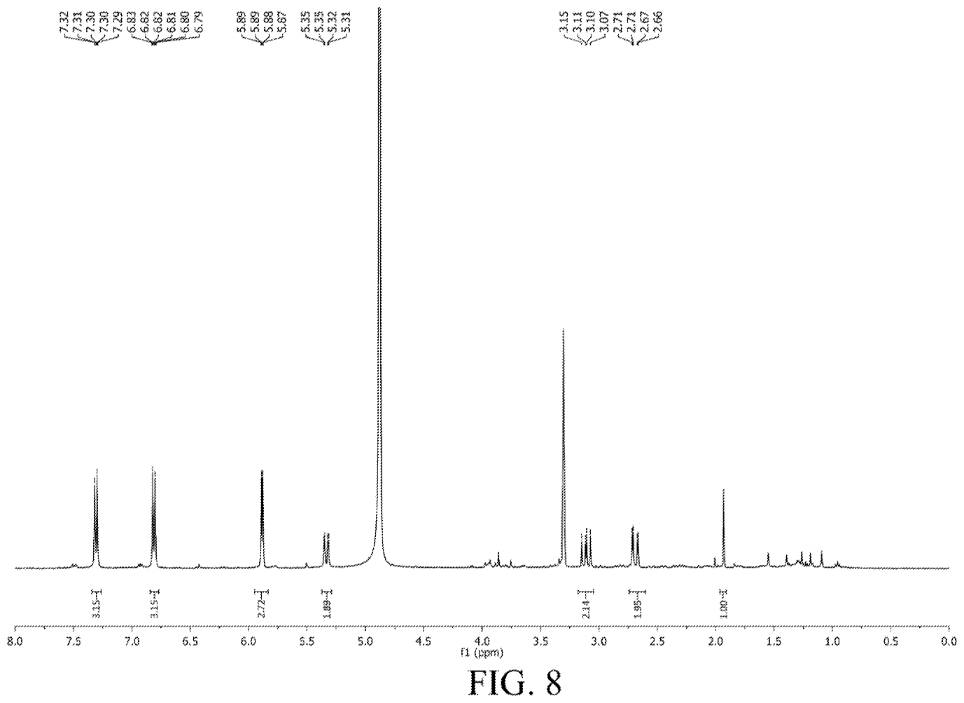

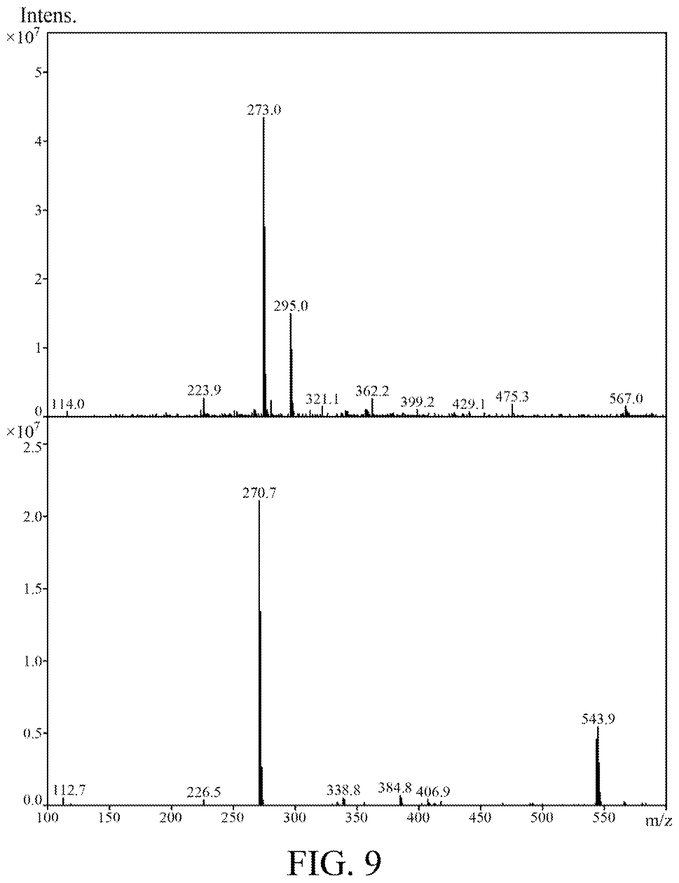

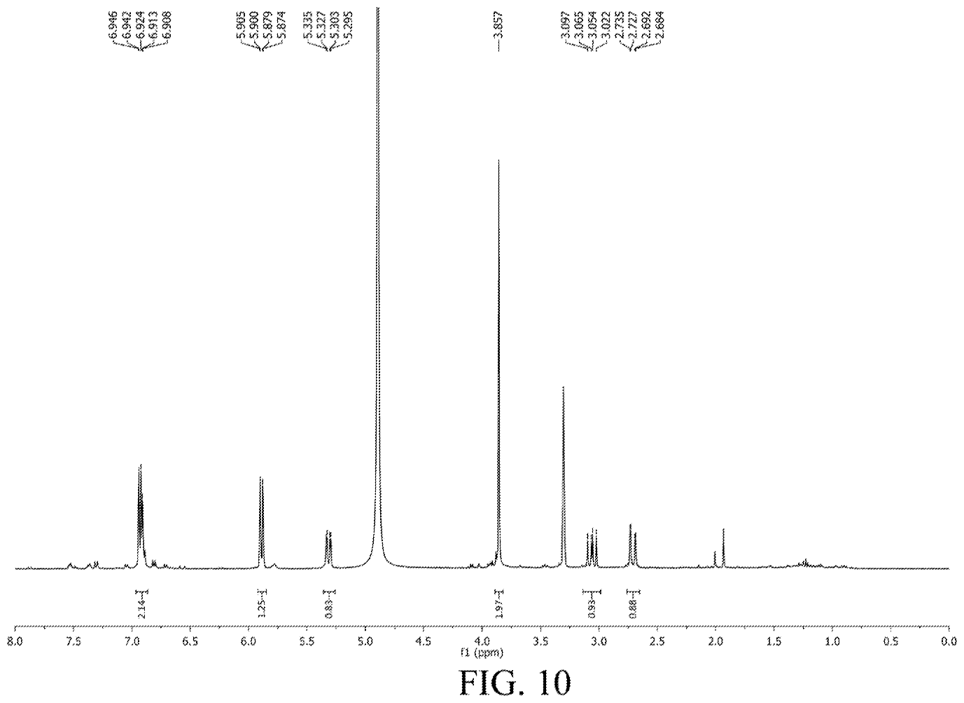

The hydrogen spectrogram data of TCI-CA-01 is shown in FIG. 2, and the mass spectrogram data of TCI-CA-01 is shown in FIG. 3. The hydrogen spectrogram data of TCI-CA-02 is shown in FIG. 4, and the mass spectrogram data of TCI-CA-02 is shown in FIG. 5. The hydrogen spectrogram data of TC-CA-03 is shown in FIG. 6, and the mass spectrogram data of TCI-CA-03 is shown in FIG. 7. The hydrogen spectrogram data of TCI-CA-04 is shown in FIG. 8, and the mass spectrogram data of TCI-CA-04 is shown in FIG. 9. The hydrogen spectrogram data of TC-CA-05 is shown in FIG. 10, and the mass spectrogram data of TC-CA-05 is shown in FIG. 1. The compound TCI-CA-01 is 4-hydroxy-3-methoxycinnamic acid (also referred to as (E)-3-(4-hydroxy-3-methoxy-phenyl) prop-2-enoic acid), also known as ferulic acid, and is a compound having a structural formula of Formula (I). The compound TCI-CA-02 is 7-[[2-O-(6-Deoxy-α-L-mannopyranosyl)-β-D-glucopyranosyl]]oxy]-2,3-dihydro-5-hydroxy-2-(4-hydroxyphenyl)-4H-1-benzopyran-4-one, known as naringin, and is a compound having a structural formula of Formula (II). The compound TCI-CA-03 is 5,7-Dihydroxy-2-(4-hydroxyphenyl) chroman-4-one, known as naringenin, and is a compound having a structural formula of Formula (III). The compound TC-CA-04 is 5-hydroxy-2-(3-hydroxy-4-methoxyphenyl)-4-oxo-3,4-dihydro-2h-chromen-7-yl 2-o-(6-deoxyhexopy-ranosyl)hexopyranoside, known as neohesperidin, and is a compound having a structural formula of Formula (IV). The compound TCI-CA-05 is (S)-2,3-Dihydro-5,7-dihydroxy-2-(3-hydroxy-4-methoxyphenyl)-4H-1-benzopyran-4-one, known as hesperetin, and is a compound having a structural formula of Formula (V).

TABLE 1
Chemical names and structural formulas of compound TCI-CA-01,
compound TCI-CA-02, compound TCI-CA-03, compound
TCI-CA-04, and compound TCI-CA-05

| Compound | Name | Structural formula |
|---|---|---|
| TCI-CA-01 | 4-hydroxy-3-methoxycinnamic acid (aiso referred to as (E)-3-(4-hydroxy-3-methoxy- phenyl)prop-2-enoic acid) | 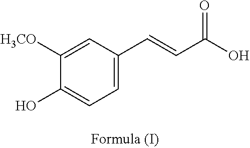 [SMILES: COc1cc(/C=C/C(=O)O)ccc1O] |
| TCI-CA-02 | 7-[[2-O-(6-Deoxy-α-L- mannopyranosyl)-β-D- glucopyranosyl]]oxy]-2,3-dihydro- 5-hydroxy-2- (4-hydroxyphenyl)-4H- 1-benzopyran-4-one | 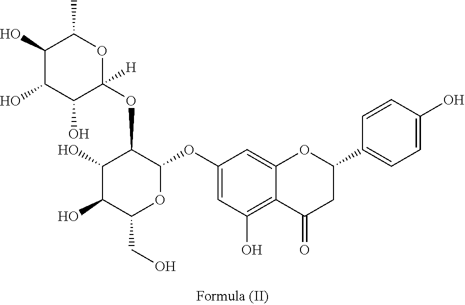 [SMILES: C[C@@H]1O[C@@H](O[C@H]2[C@H](Oc3cc(O)c4c(c3)O[C@H](c3ccc(O)cc3)CC4=O)O[C@H](CO)[C@@H](O)[C@@H]2O)[C@H](O)[C@H](O)[C@H]1O] |
| TCI-CA-03 | 5,7-Dihydroxy-2-(4-hydroxy phenyl)chroman-4-one | 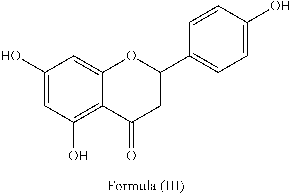 [SMILES: O=C1CC(c2ccc(O)cc2)Oc2cc(O)cc(O)c21] |
| TCI-CA-04 | 5-hydroxy-2-(3-hydroxy-4- methoxy-phenyl)-4-oxo-3,4- dihydro-2h-chromen-7-yl 2-o-(6-deoxyhexopy-ranosyl) hexopyranoside | 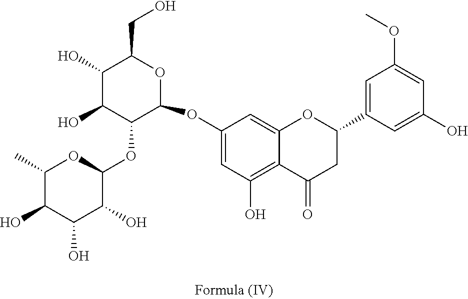 [SMILES: COc1cc(O)cc([C@@H]2CC(=O)c3c(O)cc(O[C@@H]4O[C@H](CO)[C@@H](O)[C@H](O)[C@H]4O[C@@H]4O[C@@H](C)[C@H](O)[C@@H](O)[C@H]4O)cc3O2)c1] |
| TCI-CA-05 | (S)-2,3-Dihydro-5,7-dihydroxy- 2-(3-hydroxy-4-methoxyphenyl)- 4H-1-benzopyran-4-o | 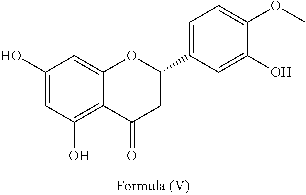 [SMILES: COc1ccc([C@@H]2CC(=O)c3c(O)cc(O)cc3O2)cc1O] |

### Embodiment 3 Efficacy of Active Ingredients of Citrus aurantium Extract for Promoting Lipolysis

According to the present embodiment, bone marrow stromal cells of mice were used for testing the efficacy of the compound TC-CA-01, the compound TCI-CA-02, the compound TC-CA-03, the compound TCI-CA-04, and the compound TCI-CA-05 for prompting lipolysis. The bone marrow stromal cells of mice were purchased from American Type Culture Collection (American ATCC®), and the No. was CRL-2749M. Before differentiation, the cells were cultured in a pre-adipocyte expansion medium, including 90 vol % of an α-MEM (Minimum Essential Medium Eagle-Alpha Modification, purchased from Gibco, No. 11095080, America) cell culture medium, and 10 vol % of fetal bovine serum (purchased from Gibco, No. 10437-028, America). In addition, 1.0 vol % of penicillin-streptomycin (purchased from Gibco, No. 15140122, America) was added. A differentiation medium was used for differentiating the bone marrow stromal cells of mice, and included 90 vol % of α-MEM cell culture medium, and 10 vol % of fetal bovine serum. In addition, 1.0 vol % of penicillin-streptomycin was added.

In order to prove the efficacy of the compound TCI-CA-01, the compound TCI-CA-02, the compound TCI-CA-03, the compound TC-CA-04, and the compound TC-CA-05 for promoting lipolysis, firstly, the bone marrow stromal cells of mice were differentiated into adipocytes. The bone marrow stromal cells of mice were inoculated in a culture tray by 8×10⁴ cells/0.5 mL of the above pre-adipocyte expansion medium in each well. Culture was performed at 37° C. for 7 days. The culture medium in each well was replaced by the above mentioned fresh differentiation medium once every 3 days during the period. After 7 days, a microscope was used to observe and confirm that the lipid droplets have formed so as to ensure the cells completely differentiated into adipocytes. Then, the cells were divided into the following seven groups: (1) a control group (Mock): which was only further added with the differentiation medium (adding amount: 10 μg differentiation medium per original volume (mL) in the well); (2) an experiment group added with the compound TCI-CA-01 (adding amount: 10 μg TCI-CA-01 per original volume (mL) in the well); (3) an experiment group added with the compound TCI-CA-02 (adding amount: 10 μg TCI-CA-02 per original volume (mL) in the well); (4) an experiment group added with the compound TCI-CA-03 (adding amount: 10 μg TCI-CA-03 per original volume (mL) in the well); (5) an experiment group added with the compound TCI-CA-04 (adding amount: 10 μg TCI-CA-04 per original volume (mL) in the well); (6) an experiment group added with the compound TCI-CA-05 (adding amount: 10 μg TCI-CA-05 per original volume (mL) in the well); and (7) an experiment group added with the compound TC-CA-02 (adding amount: 10 μg TCI-CA-02 per original volume (mL) in the well) and the compound TCI-CA-03 (adding amount: 10 μg TCI-CA-03 per original volume (mL) in the well). Culture was performed at 37° C. for 7 days. Similarly, the fresh differentiation medium was also replaced once every 3 days.

Then, a value of glycerol outside the cells was measured by a glycerol assay kit (purchased from Cayman Chemical, No. 10011725, America). After triglyceride stored in the cells was decomposed, free glycerol and fatty acid would be generated, so that a lipolysis decomposition value in the cells could be inferred by measuring the content of the glycerol outside the cells. This kit quantified the value of glycerol outside the cells by measuring the content of a product of a coupling enzyme reaction system capable of generating a bright purple, and used a glycerol standard in the kit to build a standard curve so as to reckon the value of the glycerol outside the cells of a sample. 25 μL of cell culture supernatant in each well of the 7 groups was respectively collected and was transferred into a new culture tray. Then, 100 μL of reconstructed free glycerol assay reagent in the kit was added into each well for reacting at room temperature for 15 min. Then, an OD (Optical Density) value at 540 nm was read by a spectrophotometer (Bio Tek), and student's t-test was performed by Excel software so as to determine whether significant differences statistically exist or not between two sample groups (* represents p<0.05. ** represents p<0.01. *** represents p<0.001).

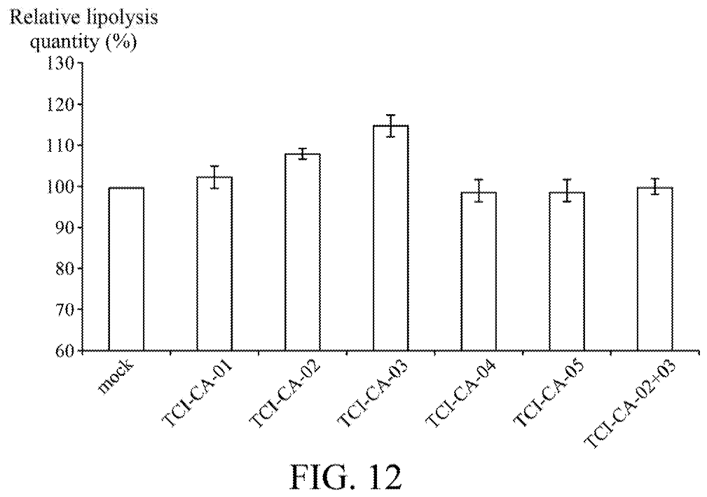

The test result of the efficacy of the compound TCI-CA-01, the compound TCI-CA-02, the compound TCI-CA-03, the compound TCI-CA-04 and the compound TCI-CA-05 of the present disclosure for promoting lipolysis was as shown in FIG. 12. As shown in FIG. 12, it can be seen that after the cells were reacted with the compound TCI-CA-01 of the present disclosure, the lipolysis could be effectively promoted in comparison with the control group; after the cells were respectively reacted with the compound TC-CA-02 and the compound TCI-CA-03 of the present disclosure, 8% and 15% of lipolysis could be effectively promoted in comparison with the control group respectively. After the cells were respectively reacted with compound TC-CA-04 and the compound TCI-CA-05 of the present disclosure, the values of these two groups were all similar to the values of the control group. This result showed that the active ingredients: the compound TCI-CA-01, the compound TCI-CA-02, and the compound TCI-CA-03 of the Citrus aurantium extract of the present disclosure could effectively promote the lipolysis so as to reduce the fat content in the cells. However, if treated the cells with the compound TCI-CA-02 and the compound TCI-CA-03 with excellent lipolysis promoting capabilities simultaneously, the lipoysis promoting efficacy was merely similar to that of the control group. This result showed that not any combination of compounds with the lipolysis promoting capability could effectively increase the lipolysis promoting efficacy. Thus, in order to obtain a better availability of active ingredients, in some embodiments, the compound TCI-CA-02 and the compound TCI-CA-03 may be not simultaneously exist in the composition.

Based on the above, in the compounds of 4-hydroxy-3-methoxycinnamic acid (also referred to as (E)-3-(4-hydroxy-3-methoxy-phenyl)prop-2-enoic acid), the 7-[[2-O-(6-Deoxy-α-L-mannopyranosyl)-β-D-glucopyranosyl]]oxy]-2,3-dihydro-5-hydroxy-2-(4-hydroxyphenyl)-4H-1-benzopyran-4-one, the 5,7-Dihydroxy-2-(4-hydroxyphenyl)chroman-4-one, the 5-hydroxy-2-(3-hydroxy-4-methoxy-phenyl)-4-oxo-3,4-dihydro-2h-chromen-7-yl 2-o-(6-deoxyhexopy-ranosyl) hexopyranoside, and the (S)-2,3-Dihydro-5,7-dihydroxy-2-(3-hydroxy-4-methoxyphenyl)-4H-1-benzopyran-4-one of the present disclosure, the 4-hydroxy-3-methoxycinnamic acid (also referred to as (E)-3-(4-hydroxy-3-methoxy-phenyl)prop-2-enoic acid), the 7-[[2-O-(6-Deoxy-α-L-mannopyranosyl)-β-D-glucopyranosyl]]oxy]-2,3-dihydro-5-hydroxy-2-(4-hydroxyphenyl)-4H-1-benzopyran-4-one, and the 5,7-Dihydroxy-2-(4-hydroxyphenyl)chroman-4-one can effectively promote lipolysis to reduce the fat content in the cells. Therefore, the 4-hydroxy-3-methoxycinnamic acid (also referred to as (E)-3-(4-hydroxy-3-methoxy-phenyl)prop-2-enoic acid), the 7-[[2-O-(6-Deoxy-α-L-mannopyranosyl)-β-D-glucopyranosyl]]oxy]-2,3-dihydro-5-hydroxy-2-(4-hydroxyphenyl)-4H-1-benzopyran-4-one, and the 5,7-Dihydroxy-2-(4-hydroxyphenyl) chroman-4-one of the present disclosure can be used to promote lipolysis and/or reduce the cell fat content. In addition, the composition is a medicine, a health care product or a food product capable of being applied to an individual in modes of oral administration, smearing, etc.

What is claimed is:

1. A method of preventing or treating obesity in a subject, wherein the method comprises administering a composition to the subject, and the composition has at least one bioactive ingredient with therapeutically effective concentration selected from the group consisting of (E)-3-(4-hydroxy-3-methoxy-phenyl)prop-2-enoic acid, 7-[[2-O-(6-Deoxy-α-L-mannopyranosyl)-β-D-glucopyranosyl]]oxy]-2,3-dihydro-5-hydroxy-2-(4-hydroxyphenyl)-4H-1-benzopyran-4-one, and 5,7-Dihydroxy-2-(4-hydroxyphenyl)chroman-4-one; and
a pharmaceutically acceptable carrier.

2. The method according to claim 1, wherein the effective concentration of the bioactive ingredient is at least 10 μg/mL.

3. The method according to claim 1, wherein the composition promotes lipolysis.

4. The method according to claim 3, wherein the composition promotes lipolysis in adipocytes.

5. A method of promoting lipolysis in a subject, wherein the method comprises administering a composition to the subject, and the composition has at least one bioactive ingredient with therapeutically effective concentration selected from the group consisting of (E)-3-(4-hydroxy-3-methoxy-phenyl)prop-2-enoic acid, 7-[[2-O-(6-Deoxy-α-L-mannopyranosyl)-β-D-glucopyranosyl]]oxy]-2,3-dihydro-5-hydroxy-2-(4-hydroxyphenyl)-4H-1-benzopyran-4-one, and 5,7-Dihydroxy-2-(4-hydroxyphenyl)chroman-4-one; and
a pharmaceutically acceptable carrier.

6. The method according to claim 5, wherein the effective concentration of the bioactive ingredient is at least 10 μg/mL.

7. The use according to claim 5, wherein the lipolysis is in adipocytes.

8. A method of preventing or treating obesity in a subject, wherein the method comprises administering a therapeutically effective amount of 7-[[2-O-(6-Deoxy-α-L-mannopyranosyl)-β-D-glucopyranosyl])oxy)-2,3-dihydro-5-hydroxy-2-(4-hydroxyphenyl)-4H-1-benzopyran-4-one to the subject.

9. The method according to claim 8, wherein the 7-[[2-O-(6-Deoxy-α-L-mannopyranosyl)-β-D-glucopyranosyl]]oxy]-2,3-dihydro-S-hydroxy-2-(4-hydroxyphenyl)-4H-1-benzopyran-4-one promotes lipolysis.
